(19) United States
(12) Patent Application Publication
(10) Pub. No.: US 2005/0181044 A1
(43) Pub. Date: Aug. 18, 2005

(54) Compositions and methods for timed release of water-soluble nutritional supplements, green coffee extract

(76) Inventor: Jaime Romero, Barranguilla (CO)

Correspondence Address: Nancy Johnson;Nancy Lord Johnson, Ltd., Suite 3, 361 South Frontage Rd., Pahrump, NV, 89048, US

(21) Appl. No.: 10/930,560

(22) Filed: Dec. 9, 2004

Related U.S. Application Data
(63) Continuation-in-part of application No. 10/782,245, filed on Feb. 18, 2004.
(63) Continuation-in-part of application No. 10/910,787, filed on Aug. 3, 2004.

(51) Int. Cl. A61K 9/20; A61K 9/22

(52) U.S. Cl. 424/464; 424/468

(57) ABSTRACT

The present invention relates to compositions of and methods for producing timed or retarded release formulations that contain green coffee extract, and optionally vinpocetine, L-camitine, of amylase inhibitor, niacin, Vitamin B3 (niacinamide), vitamin B6 (pyridoxine hydrocholoride), vitamin B12 (cyanocobaolamin), vitamin B5 (pantothenic acid), inositol, phosphorus, iodine, and chromium and/or their dietary and nutraceutically acceptable salts of the same and/or hydrates of the active substance that provide a timed or retarded release formulation of the active substance.

This is a continuation-in-part application, 37 C.F.R. 1.53(b) and claims priority based on U.S. patent application Ser. No. 10,782,245, filed Feb. 18, 2004., and Ser. No. 10,910,787 filed on Aug. 3, 2004.

### FIELD OF THE INVENTION

The present invention relates to a composition of one or more pellets for a timed or retarded release of a water-soluble nutritional supplement in the stomach and/or gastrointestinal tract of a human, comprising an admixture of an effective amount of a nutritional supplement to be released at a controlled rate and a formulation comprising a core and coating.

### BACKGROUND OF THE INVENTION

The last two decades have seen rapid development in the area of drug delivery. In particular, a number of drug delivery systems have been developed to effect the controlled release of pharmacologically active agents. For a general overview of the art, reference may be had, inter alia, to R. Baker, CONTROLLED RELEASE OF BIOLOGICALLY ACTIVE AGENTS, New York: John Wiley & Sons, 1987, Sustained Release Medications, CHEMICAL TECHNOLOGY REVIEW No. 177. Ed. J. C. Johnson. Noyes Data Corporation 1980, CONTROLLED DRUG DELIVERY, FUNDAMENTALS AND APPLICATIONS, 2nd Edition. preferred. J. R. Robinson, V. H. L. Lee. Mercel Dekkes Inc. New York 1987, REMINGTON'S PHARMACEUTICAL SCIENCES 16th Edition, Mack Publishing Company 1980, Ed. A. Osol. The term “controlled release” is intended to refer to any formulation in which release of the active substance, usually a drug, is not immediate, i.e., with a “controlled release” formulation, oral administration does not result in immediate release of the drug into an absorption pool. The term is used interchangeably with “nonimmediate release” as defined in Remington: THE SCIENCE AND PRACTICE OF PHARMACY, Nineteenth Ed. (Easton, Pa.: Mack Publishing Company, 1995).

The technology in the field of nutritional supplements, however, does not offer the plethora of controlled release delivery systems that are available for drugs. Herbs especially have not been successfully formulated for timed or retarded release. This is despite the fact that it is well known to those skilled in the art that controlled release formulations which are effective in maintaining nutraceutical blood levels over extended periods of time result in optimal supplementation.

Nutritional supplements are known as nutraceuticals when they have a proven benefit to the structure or function of a body organ or system. Studies demonstrating their benefit use the nutraceuticals at high doses, such as 500 mg-1 g. Muller-Fassbender H, et al, 2(1) OSTEOARTHRITIS CARTILAGE 61-9 (1994). Hence, a controlled release formulation that permits only, for instance, 20% of the active substance by weight would require up to 5 g total, making its use unacceptable.

A controlled release formulation that contained only small amounts of formulation components other than the nutraceutical would be desirable, not only because they reduce the frequency of dosing for enhanced user convenience and compliance, but they also reduce the severity and frequency of gastric difficulties as they maintain substantially constant blood levels and avoid the need for large and repeated doses of immediate release formulations required to achieve the benefits reported in the literature. It is, however, difficult to develop controlled release formulations of high dose nutritional supplements due to the unacceptably large sizes of the finished dosage form. While side effects are rarely seen with nutritional supplements, their severity and frequency is lessened when constant blood levels are provided as opposed to the drastic fluctuations seen with the dosing schedule.

In an effort to overcome the problem gastric irritation and to deliver glucosamine, along with a nitric oxide synthase inhibitors, U.S. Pat. Nos. 6,346,519 and 6,656,925, to Petrus, relate to, respectively, a method of treatment of arthritis and controlled release composition that includes glucosamine that consists only of a typical enteric coated tablet, preferably coated with polyvinylpyrrolidone (PVP). Enteric coatings are pH sensitive polymers designed to remain intact in the acidic environment of the stomach, but to dissolve in the more alkaline environment of the intestine. Some polymers commonly used for enteric coatings are cellulose acetate phthalate (CAP), hydroxypropyl methylcellulose phthalate (HPMCP), cellulose acetate trimellitate (CAT), hydroxypropyl methylcellulose acetate succinate (HPMCAS), polyvinyl acetate phthalate (PVAP) and acrylic resins. One formulation of the Petrus invention uses 7 mg of polyvinylpirrolidone to coat the immunostimulant composition. Disintegration of the PVP enteric coating occurs in approximately 40 minutes, about the time the composition is in the intestine. Petrus does not teach a formulation to administer a nutritional supplement over a longer interval of twelve (12) hours.

U.S. Pat. No. 5,041,292, to Feijin, relates to a biodegradable hydrogel, which has significantly enhanced biocompatibility in that (1) blood compatibility is substantially improved, (2) immunogenicity is minimized, and (3) the hydrogel is enzymatically degraded to endogenous, nontoxic compounds. The process for making the novel hydrogel allowed for control of the degree of crosslinking. By varying the composition of the hydrogel as it is made, one can control the uptake of a particular drug, the degradation kinetics of the hydrogel formulation and the overall timed-release profile. While Feijin uses chondroitin as a mucopolysaccharide in his formulation, it is not used therein as the active compound. Feijin shows that if the calcium sulfate amount is set at 1 g, the amount of drug used is in the range of 1-200 mg and the matrix biopolymer in the range of 0.4-3 ml. The concentration of the matrix biopolymer ranges from 0.1-50%, and the concentration of drug is no greater than 20%.

U.S. Pat. No. 6,649,187, to Hussain relates to further advancements in the use of polyalkylamine polymers to form hydrogel matrices for delivery of amine drugs. U.S. Pat. No. 6,630,486, to Royer shows the use of chondroitin sulfate as a glycosaminoglycan as a complexing agent, but not as the active agent. U.S. Pat. No. 6,551,620 to Otterbeck, relates to a pellet formulation having a controlled release profile for the treatment of the intestinal tract, wherein the active compound is present in the core in a non gel-forming polymer matrix selected from the group consisting of poly(ethyl acrylate, methyl methacrylate) and poly(ethyl acrylate, methyl methacrylate, trimethylammonioethyl methacrylate chloride).

U.S. Pat. No. 6,610,282, to Ghosh relates to the use of certain hydroxystyrene polymers to control the release of biologically active compounds to control or inhibit the growth of fungi, bacteria, algae, marine fouling organisms, plants, and insects.

U.S. Pat. No. 5,300,300 to Egidio relates to a controlled release formulation for the treatment of biliary tract disease that includes glucosamine as an ursodeoxycholic acid salt among the group consisting of the salt with sodium, lithium, potassium, triethylamine, triethanolamine, trimethanolamine, N-methylpiperidine, piperazine, morpholine, -methylmorpholine, 1-(2-hydroxyethyl)pyrrolidine, L-arginine, L-lysine, L-ornithine, D-glucamine, -methyl-D--glucamine, glucosamine and choline. Egidio does not teach a formulation for other larger water-soluble/low soluble nutritional supplements or herbs, nor the adjustment of the amounts of the components of the retarded release coating according the dissolution profile of the formulation.

U.S. Pat. No. 6,607,751 to Odidi, relates to a controlled release pharmaceutical device, which provides sustained or pulsatile delivery of pharmaceutically active substances for a predetermined period of time, the device comprising; about 25 to 60% by weight microbial polysaccharide; and about 15 to 60% by weight cellulose ether. While the patent claims the solution can comprise up to 80% of the pharmaceutical active, the above percentages limit it to 60% and the composition is designed for drugs, mostly cardiovascular. In another patent of Odidi, U.S. Pat. No. 6,652,882, a controlled release formulation of buproprion, a psychotropic, that utilizes 20% to about 25% by weight of an uncrosslinked polymer selected from the group consisting of hydroxyethyl cellulose, sodium carboxymethyl cellulose, hydroxypropyl cellulose, hydroxypropylmethyl cellulose, and mixtures thereof, specifies a 30% weight for the active product. Similarly, U.S. Pat. No. 6,156,342 to Sriwongjanya realtes to a pelletized controlled release form for tramadol, wherein the active drug comprises 10%-30% of the formulation.

U.S. Pat. No. 6,673,369, to Rampal, relates to a formulation of at least one drug having a water solubility of less than one part per 30 parts water, and from about 0.1% to about 4.5% w/w of one or more rate controlling high viscosity cellulosic ether polymers wherein the high viscosity polymer comprises a polymer having a viscosity of at least about 4,000 cps or more. While Rampal claims a concentration as high as 90%, Rampal's formulation requires very specific viscosity polymers, and is designed for drugs not nutritional supplements.

Several manufacturers, such as Nutravite of Canada and Contract Pharmacal of Long Island, N.Y., market glucosamine and chondroitin as gelcaps/caplets, not pellets in capsules, but the manufacturers do not provide the specific dissolution data, nor do the formulations permit the practitioner to adjust the ratio of his components according to that profile as in this invention. The present invention also teaches the formulation of pellets that can easily be administered in capsules in different amounts to provide different dosages.

Arthritis

Arthritis, a musculoskeletal disorder, is the leading cause of disability in the United States. The Centers for Disease Control and Prevention (CDC) stated that arthritis and other rheumatic conditions accounted for about 744,000 hospitalizations and 4 million days of care in 1997. Forty million Americans, representing 15% of the population, have some form of arthritis, and that figure is expected to increase to 59.4 million (18.2%) by the year 2020, an increase of 57% in the number of persons affected. Arthritis patients make more than 315 million physician visits and are hospitalized more than 8 million times a year. Arthritis costs the nation $65 billion annually in medical costs and lost productivity. Osteoarthritis (OA), or degenerative joint disease, is the most common type of arthritis, affecting 20.7 million people, (12.1%) of U.S. adults in 1990, now estimated at 37 million, and trailed chronic heart disease as the leading cause of Social Security payments due to long-term absence from work. Lawrence R C, et al. ARTHRITIS & RHEUMATISM 1998; 41:778-799.

Approximately 1-2% of the population suffers from rheumatoid arthritis (RA), which is characterized as an imbalance in the immune system that causes an overproduction of pro-inflammatory cytokines, e.g., tumor necrosis factor alpha (TNF.alpha), interleukin 1 (IL-1), and a lack of anti-inflammatory cytokines, e.g. IL-10, IL-11. RA is characterized by synovial inflammation, which progresses to cartilage destruction, bone erosion and subsequent joint deformity. The primary symptoms of RA are joint inflammation, swelling, difficulty moving, and pain. During the inflammatory process, polymorphonuclear cells, macrophages, and lymphocytes are released. Activated T-lymphocytes produce cytotoxins and pro-inflammatory cytokines, while macrophages stimulate the release of prostaglandins and cytotoxins. Vasoactive substances (histamine, kinins, and prostaglandins) are released at the site of inflammation and cause edema, warmth, erythema, and pain associated with inflamed joints.

Osteoarthritis usually presents as pain, which worsens with exercise, or simply an X-ray that clearly shows thinning cartilage. Common joints affected are the knees, hips and spine, finger, base of thumb and base of the big toe. Osteoarthritis is characterized by degenerative changes in the articular cartilage and subsequent new bone formation at the articular margins. The primary defect in hyaline cartilage, at the articular surface of the joint, is an alteration in the ratio of total glycosaminoglycans to that of the collagen fiber content in the matrix. Yasuda K. Hokkaido Igaku Zasshi July 1997;72(4):369-76. Paleontologists have found osteoarthritis to exist in almost every vertebrate. By age 60, almost all Americans have osteoarthritis in their necks or spines. Joint cartilage consists of only 5 percent cells, and joint cartilage lesions do heal. Tindall W N. Business & Health December 1997;47-48. Bones directly underneath the cartilage in joints is called subchondral bone. This bone nourishes the cartilage with oxygen, water, and nutrients conveyed through microscopic channels. This supply route carries “chondroprotective agents” from the bloodstream to the cartilage.

Cartilage is the supporting structure of the body, but has no blood vessels, nerves or lymphatics, and consists of thick bundles of fibrous protein (collagen), which are woven to form the articular surface. Proteoglycans fill the extracellular spaces not occupied by collagen, and are a combination of protein and sugar. Each proteoglycan subunit contains a protein core attached to hundreds of long chains of specially modified sugars called glycosaminoglycans (GAGs). Glucosamine is the single most important component and precursor for GAGs. Glucosamine is almost completely absorbed by the GI tract into the bloodstream. Cartilage rebuilding relates directly to GAG synthesis. Chondrocytes in the cartilage obtain glucosamine from the subchondral blood vessels and manufacture N-acetylglucosamine (NAG) and glucuronic acid, which make hyaluronan, which is half glucosamine, and provides the lubricating ability of joints. There is no definitive answer regarding the cause of osteoarthritis. A natural erosion of cartilage occurs with age, but excessive loads placed on joints, obesity, heredity, trauma, decreased circulation, poor bone alignment, and repetitive stress motion play a role. Osteoarthritis may also be the result of free radical damage, thought to be a major cause of many diseases, including the aging process, cancer, heart disease and degenerative diseases.

Glucosamine

Glucosamine from exogenous sources (food and supplements) may stop the progression of cartilage degradation and stimulate the production of new cartilage. Glucosamine absorbed by the gastrointestinal tract undergoes significant first-pass metabolism in the liver, with the resulting 26% bioavailability. It is incorporated into plasma proteins as a result of hepatic metabolism, and concentrates in the articular cartilage. Clinical improvement of symptoms has been seen as early as one week after oral administration of glucosamine sulfate and has persisted for up to four weeks after discontinuation. Barclay T S, Tsourounis C, McCart G M. Glucosamine. ANNALS OF PHARMACOTHERAPY 1998;32:574-79. A three-year study of 212 patients with knee osteoarthritis showed long-term combined with structure-modifying and symptom-modifying effects of glucosamine sulfate. Reginster J Y, et al, LANCET 2001;357:251-56. Oral glucosamine was demonstrated to be utilized in newly synthesized proteoglycan and ended up in the cartilage matrix. Noyszewski E A, et al, ARTHRITIS & RHEUMATISM 2001;44:1089-1095.

Several commercial forms of glucosamine are available, including the sulfate, hydrochloride, N-acetylglucosamine (NAG). Glucosamine hydrochloride has a higher concentration of glucosamine than the sulfate form. NAG is rapidly metabolized to make proteins and provides less glucosamine for cartilage repair. The composition of the invention could include one or a combination of the glucosamine forms. Patients have reported a more rapid response with higher dosages of glucosamine, but the therapeutic results with glucosamine alone have not been consistent. The dosage range for glucosamine can vary from 100 mg to 3000 mg a day, in divided doses, depending on body weight and severity of symptoms. One approach is to take 1,500 mg of glucosamine daily until symptoms have decreased, then reduce the dosage to 1,000 mg for two weeks and eventually stop treatment after symptoms cease or stay on a maintenance dose of 500 mg per day.

Adverse effects reported from glucosamine are gastrointestinal, such as heartburn and epigastric pain. Because the half-life of glucosamine in the blood is relatively short, a sustained-release form of the compound could avoid the adverse effects and provide a more uniform blood level, Talent J. M, Gracy R W. CLINICAL THERAPY 1996; 18(6):1184-90.

Chondroitin

Chondroitin, the glucosamine found in cartilage, is also used extensively in both the treatment of arthritis and for nutritional support of healthy bones and joints. Richy F, et al, ;163(13) ARCH INTERN MED. 1514-22 (2003). Like glucosamine, it is generally used in dosages between 100 mg and 3000 mg per day, preferably in the range of 200 mg to 600 mg, most preferably 500 mg.

The chemical formula for chondroitin is: (C₁₄H₁₉NO₄SNa₂)ₙ; N-acetylchondrosamine (2-acetamide-2-deoxi-D-galactopiranose) and D-gluoronic acid copolymer. Its physiologic function is to increase the lubrication of the articulations, which gives them more freedom of movement and helps them transfer fluids to the cartilage. The Chondroitin also affects the capillary vessels, relieves obstructions improving circulation and reduces excessive coagulation.

Green Coffee Extract

The improved attention and energy effected by drinking coffee are well established both culturally and scientifically. Caffeine is a natural constituent of the coffee plant and can be isolated from coffee. It is believed to effect the release of dopamine, which is reduced in fatigue. Numerous authors have demonstrated the effects of coffee, or synthetic caffeine, using objective psychometric tests of performance. Reyner L A, and Home, J A 75(3) PHYSIOL BEHAV. 331-5 (2002); Lieberman, H R et al, 164(3) PSYCHOPHARMACOLOGY (Berl) 250-61 (2002); Beaumont, M, et al, 10(4) J SLEEP RES. 265-76 (2001). The addition of taurine to a functional energy drink containing only 80 mg of caffeine has shown to improve the effectiveness of that drink over a 300 mg caffeine in studies of driver simulation. Lorist, M M and Tops, M, 53(1) BRAIN COGN. 82-94 (2003). Slow release synthetic caffeine has been shown to have excellent effects. De Valck E, De Groot E, and Clydts R, 91 (6) PERCEPT MOT SKILLS. 67-78 (2003). Coffee contains numerous minerals not found in synthetic caffeine, including chlorogenic acid, boron, silicon, chromium, barium, tungsten, and bismuth.

Several inventors have disclosed methods for preparing a controlled release formula containing synthetic caffeine, but these do not provide the benefit of whole coffee extract. U.S. Pat. No. 6,491,950, to Gutierrez-Rocca, discloses a such a method using a matrix construct of a component selected from a high melting point fatty acid ester, an oil, a polymeric cellulose derivative, and a mixture of these, along with numerous medicines including caffeine. U.S. Pat. Nos. 5,744,164 and 5,700,484, to Chauffard, disclose sustained release microparticulate caffeine formulation that requires a mixture of a binding agent and a water releasing agent with the caffeine in the first layer applied to the core, and a second layer of insoluble material, and a third layer of active compound mixed with binding agent and water releasing agent in a third layer. Chauffard states that caffeine can be obtained from coffee beans, tea leaves, cola nuts, guarana, cacao seeds, or mate, or synthetic caffeine or other methyl-xanthines be utilized. Chauffard's method is performed 60 degrees C., and results in 10 hour dissolution profile in which 20-50% is released in 2 hours.

### SUMMARY OF THE INVENTION

The need exists for a timed or retarded release nutraceutical composition that provides a longer delay and a high concentration of a nutritional supplement whose dissolution allows greater flexibility in designing timed release profiles, provides improved plasma levels, and is simply and economically produced. Such a delayed delivery dosage form has a practical application, and it represents a valuable contribution to the medical arts. The present invention provides such a composition, and offers an efficient and cost effective method of preparation. Accordingly, it is an object of this invention to provide a timed or retarded release formulation of water-soluble nutritional supplements, including herbs, suitable for twice daily administration.

Another object of the present invention is to provide a capsule dosage form comprising means for delaying delivery of the drug for up to twelve (12) hours. To obtain a dissolution profile under twelve hours, the practitioner reduces the amount of the coating agents relative to those that comprise the core of the formulation. Using this methodology, complete dissolution in eight (8) hours or other selected intervals may be obtained.

It is also an object of this invention to provide a timed and retarded release glucosamine capsule formulation that is easy to manufacture and can be used to prepare a range of dosing levels suitable for administration. The present invention meets the unfulfilled needs of the nutraceutical industry.

It is yet another object of this invention to provide a timed and retarded release glucosamine or chondroitin capsule formulation that is easy to manufacture and can be used to prepare a range of dosing levels suitable for twice daily administration.

The current invention involves a new pelletization process, typified by the application of a water-soluble supplement/cellulose ether suspension to inert spheres and a unique formulation of timed or retarded release coating, which is applied to separate active supplement pellets. The formulation functions by membrane-controlled extended-release in a pH dependent manner. The invention employs several components that are inactive in that they are biologically inert and in some embodiments of the invention add only volume whereas in another embodiment, certain of these components slow the release of the active compounds when used as coating agents. The components used in this invention include (1) a saccharide, preferably refined sucrose, (2) an excipient, preferably silicon dioxide, (3) a lubricant, preferably talc, (4), an agglutinative, preferably hydroxypropylmethylcellulose, (5) a stabilizer, preferably shellac gum, and (6) a plasticizer, preferably methacrylic acid co-polymer. Coating agents, which will act as retarding agents in that they slow the release of the active ingredients, are in a specific embodiment, most specifically polymers such as hydroxypropylmethylcellulose or methacrylic polymer, but may also be selected from other agglutinatives and stabilizers such as shellac gum and polyvinyl pyrrolidone. The use of these components by this methodology allows a flexibility in the formulation wherein the dissolution profile obtained by this method guides the practitioner to adjust the amounts of the various components, particularly the polymeric coating agents, to obtain the desired dissolution profile.

Numerous coating agents are known in the art and have been used with varying results including acrylics, Aquacoat®, Aquaterics®, caseinates, cellulosics, chlorinated rubber, Cateric®, coating butters, Daran®, Latex®, dextrins, enterics, Eudragits®, eva, fats, fatty acids, gelatin, glycerides, gums, halocarbons, vegatables, halocarbon resins, Kynar®, maltodextrins, microcrystalline wax, milk solids, molasses, nylon, Opadry®, parafin waxes, phenolics, polyamino acids, polyethylene, polyethylene glycol, polylactides, polyvinyl acetate, polyvinylacetate phthalate, polyvinyl alcohol, polyvinyl chloride, polyvinylidine chloride, polyvinyl pyrollidone, proteins, synthetic rubber, shellac, silicone, surfactants, starches, stearines, sucrose, Surelease®, Teflon®, waxes and zein. Multiple-units tend to be developed as controlled-release pellets by using an appropriate film coating. Two groups of polymers are commonly used for the coating: derivatives of acrylic acid such as Metha Acrylic acid, and cellulose derivatives such as ethyl cellulose such as Aquacoat® and Surelease®.

The flexible use of these components by this methodology allows formulation of the present invention to a desired dissolution profile as the practitioner can adjust the amounts of the various components, particularly the polymeric coating agents, to obtain the desired dissolution profile.

In a specific embodiment of the invention, inert pellets are prepared with a saccharide, specifically refined sugar, an excipient, specifically silicon dioxide, a lubricant, specifically talc, and an agglutinating agent, specifically hydroxypropylmethylcellulose. In a specific embodiment, the pellets are initially coated with an active ingredient, most specifically a nutritional supplement and hydroxypropylmethyl cellulose. Generally, the weight ratio may preferably be the nutritional supplement in an amount of about 88% by weight; the saccharide in an amount of about 5% by weight; the excipient in an amount of about 1.8% by weight; the lubricant in an amount of about 0.22% by weight; the agglutinative in an amount of about 1.0% by weight; a stabilizer in an amount of about 3.66% by weight; the plasticizer in an amount of about 0.35% by weight. In a specific embodiment, preferably, the nutritional supplement used in this formulation is glucosamine.

In yet another specific embodiment, the active ingredient may be chondroitin, or nutraceutically acceptable salt, ether, ester, acid or derivative thereof, present in from about 40% to 90% by weight, and in a specific embodiment most preferably between about 75% to about 88% by weight of the finished pellet. The preparation may generally contain in a specific embodiment, referring to the individual dose, from about 100 mg to about 2000 mg of chondroitin, and more specifically from about 200 mg to about 500 mg.

This inventor has discovered a timed or retarded release formulation that permits the release of large water-soluble nutritional supplements over about an interval of twelve (12) hours and can be adjusted by adjusting the ratio of the components, by weight, in accordance with the dissolution profile. It is an object of the present invention to provide a controlled release formulation suitable for twice-daily administration, comprising a nutraceutically effective amount of a water-soluble nutritional supplement.

It is further an object of the invention to provide a composition of one or more pellets for a timed or retarded release of a water-soluble nutritional supplement in the stomach and/or gastrointestinal tract of a human, comprising an admixture of an effective amount of a nutritional supplement to be released at a controlled rate and a formulation comprising the components (1) a saccharide, (2) an excipient, (3) a lubricant, (4), an agglutinative, (5) a stabilizer, and (6) a plasticizer.

Yet another object of the invention is to provide a method for the producing composition of one or more pellets for a timed or retarded release capsule dosage of a water-soluble nutritional supplement form comprising at least one controlled release pellet. The first step in this method is to weigh the water-soluble nutritional supplement and the formulation components such that the following proportions are present by weight: the nutritional supplement is present in an amount of about 60% to about 95% by weight; the saccharide is present in an amount of about 1.5% to about 15% by weight; the excipient is present in an amount of about 0.6% to about 6% by weight; the lubricant is present in an amount of about 0.07% to about 1% by weight; the agglutinative is present in an amount of about 0.3% to 3% by weight; a stabilizer is present in an amount of about 1% to about 10% by weight; and the plasticizer is present in an amount of about 0.1% to about 1% by weight.

After the components are weighed, a solution is prepared with the agglutinative. A mixture of the excipient and about half of the lubricant is prepared. The mixture of the excipient and lubricant is added to the saccharide and about one half of said solution of the agglutinative. The mixture is then formed into pellets and the pellets are dried in a drying stove and dried. The water-soluble nutritional supplement is applied using the remainder of the agglutinative solution.

After the application is completed, the pellets are and are dried in the drying stove. A solution is then prepared using a stabilizer, in a specific embodiment the stabilizer may be preferably Shellac gum, plasticizer, and the other half of the lubricant. The solution of the stabilizer, plasticizer and lubricant is then applied to the pellets to form the timed or retarded release pellets. The timed or retarded release pellets are dried and assays are performed on the pellets and the timed or retarded release pellets in a solution that mimics gastric pH. Assay is used to adjust the amounts of the formulation components to attain the desired timed or retarded release.

A further object of the invention is to provide a composition for the timed or retarded release of glucosamine sulfate wherein after 1 hour about 10% to about 30% of the nutritional supplement is released; after 4 hours about 50% to about 75% of the nutritional supplement is released; and after 8 hours about 75% to about 95% is released and at 12 hours, about 80% to about 100% of the nutritional supplement is released. This embodiment also provides methods for treating arthritis and maintaining healthy bones and joints by administering the timed or retarded release glucosamine composition in dosages between 100 mg and 2000 mg per day. This embodiment also provides methods for treating arthritis and maintaining healthy bones and joints by administering the timed or retarded release glucosamine composition in dosages between about 100 mg and about 2000 mg per day.

Yet another object of the invention is to provide a composition for the timed or retarded release of chondroitin wherein after 1 hour about 15% to about 35% of the nutritional supplement is released; after 4 hours about 45% to about 75% of the nutritional supplement is released; and after 8 hours about 75% to about 95% of the nutritional supplement is released and after 12 hours about 80% to about 100% of the nutritional supplement is released. This embodiment also provides methods for treating arthritis and maintaining healthy bones and joints by administering the timed or retarded release chondroitin composition in dosages between about 100 mg and about 2000 mg per day.

This inventor has discovered a method where it is not longer necessary to extract the caffeine from coffee or another plant for preparation of a controlled release formulation. An extract is prepared of the green coffee bean itself. In this method, the coffee extract is the water-soluble nutritional supplement and in this embodiment, specifically Shellac gum, may be used as both the agglutinative and the stabilizer, and the coffee extract is present in an amount of about 40% to about 90% by weight. Only one additional component, the agglutinative, is mixed with the coffee extract to form the active layer and no additional layer of coffee is required. This invention differs further, and offers improvements over the teachings of Chauffard, U.S. Pat. Nos. 5,744,164 and 5,700,484, in that this inventor's process is performed at room temperature and the dissolution profile extends to twelve (12) hours and only 25% of the green coffee extract is released within the first hour, and 25%-50% is released within four (4) hours.

Additional layers may be prepared of other ingredients that are known to enhance energy or improve weight loss, specifically vinpocetine, L-carnitine, of amylase inhibitor, niacin, Vitamin B3 (niacinamide), vitamin B6 (pyridoxine hydrocholoride), vitamin B12 (cyanocobaolamin), vitamin B5 (pantothenic acid), inositol, phosphorus, iodine, and chromium. In an alternative embodiment of this invention, the optional ingredients may be mixed with the green coffee extract in a single active layer.

The compositions of the invention comprise green coffee extract, combined with vinpocetine or L-carnitine, to enhance energy and produce a wakeful, yet tranquil state. The composition also had the unforseen benefit of suppressing appetite.

### DETAILED DESCRIPTION OF THE INVENTION

Definitions:

By the term “nutritional supplement” or “nutraceutical” as used herein is meant any chemical material or compound suitable for administration which induces a desired effect on the structure or function of a body organ or system. In general, this includes plant derivatives, raw plant parts, such as leaf, root, seed, and animal extracts such as glucosamine and chondroitin, and pro-hormones such as androstendiol, along with synthetic molecules that have been used historically as nutritional supplements, such as dimethylaminoethanol (DMAE). By “effective” amount of a pharmacologically active agent or drug is meant a nontoxic but sufficient amount of a compound to provide the desired systemic or local effect.

By “nutraceutically acceptable,” such as in the recitation of a “nutraceutically acceptable carrier,” or a “nutraceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained.

“Pharmacologically active” (or simply “active”) as in a “pharmacologically active” derivative or metabolite, refers to a derivative or metabolite having the same type of pharmacological activity as the parent compound and approximately equivalent in degree. When the term “nutraceutically acceptable” is used to refer to a derivative (e.g., a salt) of an active agent, it is to be understood that the compound is pharmacologically active as well, i.e., therapeutically effective to improve the function of the bones and joints.

As used herein, “nutraceutically acceptable salts” refer to derivatives of the disclosed compounds wherein the amine drug or polyalkylamine polymer is modified by making an acid salt thereof. Examples of nutraceutically acceptable salts include, but are not limited to, mineral or organic acid salts of the drug. The phrase “nutraceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Lists of suitable salts are found in texts such as REMINGTON'S PHARMACEUTICAL SCIENCES, 18th Ed. (Alfonso R. Gennaro, ed.; Mack Publishing Company, Easton, Pa., 1990); REMINGTON: THE SCIENCE AND PRACTICE OF PHARMACY 19th Ed. (Lippincott, Williams & Wilkins, 1995); HANDBOOK OF PHARMACEUTICAL EXCIPIENTS, 3rd Ed. (Arthur H. Kibbe, ed.; Amer. Pharmaceutical Assoc., 1999); the PHARMACEUTICAL CODEX: PRINCIPLES AND PRACTICE OF PHARMACEUTICS 12th Ed. (Walter Lund ed.; Pharmaceutical Press, London, 1994); THE UNITED STATES PHARMACOPEIA: THE NATIONAL FORMULARY (United States Pharmacopeial Convention); and GOODMAN AND GILMAN'S: THE PHARMACOLOGICAL BASIS OF THERAPEUTICS (Louis S. Goodman and Lee E. Limbird, eds.; McGraw Hill, 1992), the disclosures of which are hereby incorporated by reference.

By an “effective” amount or a “nutraceutically effective amount” of a nutritional supplement or pharmacologically active agent is meant a nontoxic but sufficient amount of the agent to provide the desired effect, e.g. with chondroitin, an improvement in the function of afflicted bones and/or joints.

It must be noted that as used in this specification and the appended claims, the singular forms “a,” “an” and “the” include plural referents unless the context clearly dictates otherwise. Thus, for example, reference to “an active agent” or “a nutraceutically active agent” includes a single active agent as well a two or more different active agents in combination, reference to “a lubricant” includes mixtures of two or more lubricants as well as a single lubricant, and the like.

Compositions

The present invention relates to compositions of one or more pellets for a timed or retarded release of a water-soluble nutritional supplement in the stomach and/or gastrointestinal tract of a human, comprising an admixture of an effective amount of a nutritional supplement to be released at a controlled rate in a formulation comprising the components (1) a saccharide, (2) an excipient, (3) a lubricant, (4), an agglutinative, (5) a stabilizer, and (6) a plasticizer. In one embodiment of the invention, the nutritional supplement is present in an amount of about 60% to about 95% by weight; the saccharide is present in an amount of about 1.5% to about 15% by weight; the excipient is present in an amount of about 0.6% to about 6% by weight; the lubricant is present in an amount of about 0.07% to about 1% by weight; the agglutinative is present in an amount of about 0.3% to 3% by weight; the stabilizer is present in an amount of about 1% to about 10% by weight; and the plasticizer is present in an amount of about 0.1% to about 1% by weight.

In a specific embodiment of the invention, the nutritional supplement is present in an amount of about 75% to about 95% by weight; the saccharide is present in an amount of about 3% to about 8% by weight; the excipient is present in an amount of about 1% to about 3% by weight; the lubricant is present in an amount of about 0.15% to about 0.5% by weight; the agglutinative is present in an amount of about 0.6% to 1.5% by weight; the stabilizer is present in an amount of about 2% to about 5% by weight; and the plasticizer is present in an amount of about 0.2% to about 0.5% by weight.

In a specific the most preferred embodiment of the composition, the nutritional supplement is present in an amount of about 88% by weight; the saccharide is present in an amount of about 5% by weight; the excipient is present in an amount of about 1.8% by weight; the lubricant is present in an amount of about 0.22% by weight; the agglutinative is present in an amount of about 1.0% by weight; the stabilizer is present in an amount of about 3.66% by weight; and the plasticizer is present in an amount of about 0.35% by weight.

In an alternative specific embodiment, the nutritional supplement is present in an amount of about 60% to 95% by weight; the saccharide is present in an amount of about 1.5% to about 15% by weight; the excipient is present in an amount of about 0.6% to about 6% by weight; the lubricant is present in an amount of about 0.3% to about 3% by weight; the agglutinative is present in an amount of about 0.3% to about 3% by weight; and the plasticizer is present in an amount of about 1.5% to about 12% by weight.

In an alternative specific embodiment of the invention, the nutritional supplement is present in an amount of about 75% to about 95% by weight; the saccharide is present in an amount of about 3% to about 8% by weight; the excipient is present in an amount of about 1% to about 3% by weight; the lubricant is present in an amount of about 0.15% to about 0.5% by weight; the agglutinative is present in an amount of about 0.6% to 1.5% by weight; and the plasticizer is present in an amount of about 2% to about 6% by weight.

In an alternative specific embodiment of the composition, the nutritional supplement is present in an amount of about 88% by weight; the saccharide is present in an amount of about 5% by weight; the excipient is present in an amount of about 1.8% by weight; the lubricant is present in an amount of about 0.22% by weight; the agglutinative is present in an amount of about 1.0% by weight; and the plasticizer is present in an amount of about 4% by weight.

In a specific embodiment, ideally, the composition is in the form of multiple pellets and said pellets are inside a gel capsule, either soft or hard gel. Most preferably, natural or natural color capsules of the size 590 um to 1190 um are used, which are available from vendors well known to those skilled in the art.

In a specific embodiment, the compositions of the present invention may be used for any type of water-soluble nutritional supplement, including herbal products such as the leaf, root, or extract of a plant selected from the group consisting of artichoke, bilberry, bioflavonoid, boswella, bupleurium, chamomile, chlorophyll, cranberry, damiana, echinacea, essiac, garcinia cambogia, garlic, germanium, ginger, gingko, ginseng, goldenseal, grape seed, green tea, hawthorne berry, hesperidin, hops, hydrangea, hypericum, indole-3-carbinol, licorice, lycopene, nettle root, peppermint, periwinkle, policosanol, psyllium, pygeum, quercetin, raspberry, resveratol, rutin, sassafras, saw palmetto, silymarin, tribulus terestris, turmeric, valerian, wild yam; and their nutraceutically acceptable salts, ethers, esters, acid, or other derivatives.

The composition may also be prepared with a water-soluble nutritional supplement is selected from one or more of the group consisting of an amino acid, vitamin, or animal product selected from the group consisting of acetyl-1-carnosine, alpha lipoic acid, amylase, androstendiol, androstendione, arginine, ascorbic acid, B vitamin, beta-carotene, biotin, bromelain, calcium, chicken collagen, chitosan, choline, chondroitin, coenzyme Q10, creatine, dehydroepiandrosterone, diethylmethylaminoethanol, dihydroepiandsterone, dimethylglycine, DMSO, gammahydroxybutric acid (GABA), glucosamine, glutamine, glutathione, hyaluronic acid, hydroxytryptophan, indium, isoleucine, 1-carnitine, lactoferrin, lecithin, leucine, lipase, lumbrokinase, lutein, magnesium, melatonin, Methylcobalamin, methylsulfonylmethane, MGN 3, ornithine, pancreatin, panthethoic acid, papain, para-amino benzoic acid (PABA), phenylalanine, phosphatidylcholine, phosphatidylserine, potassium, pregnenalone, protease, retinoic acid, retinol, s-adenosyl-methionine , selenium, taurine, theanine, thymase, tocopherol, trimethylglycine, tryptophan, tyrosine, valine, vitamin D, vitamin A, zeathanthine, zinc.; and their nutraceutically acceptable salts, ethers, esters, acid, or other derivatives.

In its most preferred specific embodiment, the water-soluble nutritional supplement may be glucosamine sulfate, and its their nutraceutically acceptable salts, ethers, esters, acid, other derivatives.

The saccharide may comprise a refined sugar derived from beet sugar, brown sugar, cane sugar, caramel, caramelized sugar, corn sugar, granulated sugar, or a simple saccharide such as fructose, monosaccharides or disaccharides, such as galactose, lactose, trehalose, sucrose mannose, maltose, ribose, xylose, and arabinose, all of which are readily available in bulk from manufacturers known to those skilled in the art, such as Polymer Laboratories, Inc., of Amherst, Mass.

The excipient may comprise silicon dioxide, microcrystalline cellulose, calcium phosphate, calcium sulfate, sodium laurel sulfate and silicified microcrystalline cellulose and silicon dioxide, and the like. Various silicones are available from manufacturers known to those skilled in the art, such as United Chemical Technologies, Inc., of Bristol, Pa. In a specific embodiment, the excipient may be silicon dioxide.

The lubricant may comprise be magnesium stearate, talc, or any suitable lubricant known to those of ordinary skill in the art available from numerous manufacturers including Bioclean Impex of India. In a specific embodiment, the lubricant may be talc.

The agglutinative may comprise one of the polyacrylates, polymethacrylates, polyvinylpyrrolidone, poly(vinyl acetate), various starches, corn products such as amaizo, amylose and zein, pectin, alkoxylated celluloses, polyesters, polyethers, polyethylene glycol, proteins, nucleic acids, albumin, gelatin, starch, collagen, dextran and modified dextrans, polysaccharides, polylactide/polyglycolide, polyalkylcyanoacrylates, polyacrylamide, polysorbates, polyethylene ethers and esters, and polyoxyethylene/polyoxypropylene block polymers, cellulose acetophthalate, hydroxypropylmethyl cellulose phthalate, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, cellulose acetate butyrate, methyl cellulose, ethyl cellulose, hydroxyethyl cellulose, propyl cellulose, hydroxypropyl cellulose, lower-substituted hydroxypropyl cellulose, carboxymethyl cellulose, hydroxypropylmethyl cellulose, and combinations thereof. These may also be purchased from manufacturers such as Polymer Laboratories, Inc., of Amherst, Mass., Heterene, Inc., of Paterson, N.J., Klucel.R™. from Nippon Soda, Japan and Dow Chemical Company, .S.A. and others known to those skilled in the art. In a specific embodiment, the agglutinative may be hydroxypropylmethyl cellulose.

The stabilizing agent may comprise shellac, shellac gum and its constituent aliphatic polyhydroxy acids, ascorbic acid, benzoic acid and fumaric acid. In a specific embodiment, the stabilizing agent may comprise, preferably Shellac gum. These components are readily available from several manufacturers in India such as Tolaram of Calcutta, India, and others known to those skilled in the art.

The plasticizer may comprise adipate, azelate, enzoate, citrate, stearate, isoebucate, sebacate, triethyl citrate, tri-n-butyl citrate, acetyl tri-n-butyl citrate, citric acid esters, triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate and diethylphthalate, preferably diethylphthalate. Diethylphthalate is available from Parchem of White Plains, N.Y. and other manufacturers known to those skilled in the art.

In a specific embodment, the compositions of the present invention comprises a core comprising the nutritional supplements and a coating that controls its release in a timed or retarded manner. In one embodiment, the core comprises about 62% to about 99% of a water-soluble nutritional supplement; about 1.5% to about 16% of a saccharide; about 0.65% to about 6.5% of an excipient; about 0.05% to about 0.5% of a lubricant; and about 0.3% to about 3% of an agglutinative. The semipermeable coating may comprise about 20% to about 80% of a lubricant; about 25% to about 90% of the stabilizer; about 1.5% to about 15% of a plasticizer. In a specific embodiment, the core comprises: about 78% to about 99% of a water-soluble nutritional supplement; about 3% to about 8.3% of a saccharide; about 1% to about 3.3% of an excipient; about 0.05% to about 0.5% of a lubricant; about 0.6% to about 1.6% of an agglutinative; said semipermeable coating covering said core comprises: about 30% to about 50% of a lubricant; about 40% to about 60% of the stabilizer; about 3% to about 10% of a plasticizer. In a specific embodiment, the nutritional supplement comprises glucosamine. In an alternative specific embodiment, the sacharide in the core is replaced with cellulose, which is useful for people who have difficululty metabolizing sugar.

In a specific embodiment, the core may comprise about 92% of a water-soluble nutritional supplement, preferably glucosamine; about 5% of a saccharide; about 2% of an excipient; about 0.1% of a lubricant; about 1% of an agglutinative; said semipermeable coating covering said core comprises: about 42% of a lubricant; about 53% of the stabilizer; and about 5% of a plasticizer.

In an alternative specific embodiment, the compositions of the present invention comprises a core comprising the nutritional supplements and a coating that controls its release in a timed or retarded manner. In one embodiment, the core comprises about 62% to about 99% of a water-soluble nutritional supplement; about 1.5% to about 15% of a saccharide; about 0.65% to about 6.5% of an excipient; about 0.05% to about 0.5% of a lubricant; and about 0.3% to about 3% of an agglutinative. The semipermeable coating may comprise about 60% to about 99% of a plasticizer and about 0.5% to about 7% of a lubricant. In another specific embodiment, the core comprises: about 78% to about 99% of a water-soluble nutritional supplement; about 3% to about 8.3% of a saccharide; about 1% to about 3% of an excipient; about 0.07% to about 0.3% of a lubricant; about 0.6% to about 1.6% of an agglutinative; said semipermeable coating covering said core comprises: about 80% to about 98% of said plasticizer and about 1% to about 3% of said lubricant. In a specific embodiment, the nutritional supplement comprises chondroitin.

In a specific embodiment, the core may comprise about 92% by weight of said water-soluble nutritional supplement, preferably chondroitin, about 5% by weight of said saccharide; about 2% by weight of said excipient; about 0.1% by weight of said lubricant; about 1% by weight of said agglutinative; and said semipermeable coating surrounding said core comprises about 97% by weight of said plasticizer and about 2.25% by weight of said lubricant.

In a third specific embodiment, the water-soluble nutritional supplement may be methylsulfonylmethane, and its nutraceutically acceptable salts, ethers, esters, acid and other derivatives. In this specific embodiment, the core may comprise about 92% by weight of said methylsulfonylmethane, about 5% by weight of said saccharide, or alternatively said cellulose; about 2% by weight of said excipient; about 0.1% by weight of said lubricant; about 1% by weight of said agglutinative; and said semipermeable coating surrounding said core comprises about 97% by weight of said plasticizer and about 2.25% by weight of said lubricant.

This composition may also be ideally suited for multiple pellets to be placed inside a hard gel capsule or other suitable capsule or carrier.

In a specific embodiments, the timed or retarded release dosage form pellet may exhibit the following dissolution profiles when tested in a No.2 (paddle) at 50 rpm in 900 ml of water at 37 degree C. +/−0.5 degree:

| | Embodiment 1 - | Embodiment 1 - | Embodiment 2 - | Embodiment 2 - | Embodiment 3 - | Embodiment 3 - | Embodiment 3 - |
|---|---|---|---|---|---|---|---|
| Hour | mean | range | mean | range | mean | range a | range b |
| 1 | about 19.50% | about 10 - to | about 30.46% | about 15 to | about 5.58% | less than | less than |
| | | about 30% | | about- 35% | | about 20% | about 30% |
| 4 | about 59.80% | about 50-75% | about 56.85% | about 55-75% | about 38.55% | about 20-55% | about 10% to |
| | | | | | | | about 65% |
| 8 | about 81.50% | about 75-95% | about 88.57% | about 75-95% | about 84.98% | about 55-90% | about 45% to |
| | | | | | | | about 95% |
| 12 | about 88.40% | about 80-100% | about 96.15% | about 80-100% | about 94.18% | more than | more than |
| | | | | | | about 90% | about 80% |

The invention further provides methods of producing a composition of one or more pellets for a timed or retarded release capsule dosage of a water-soluble nutritional supplement form comprising at least one controlled release pellet. First, the water-soluble nutritional supplement and the formulation components are weighed such that the following proportions are present by weight: the nutritional supplement is present in an amount of about 60% to about 95% by weight; the saccharide is present in an amount of about 1.5% to about 15% by weight; the excipient is present in an amount of about 0.6% to about 6% by weight; the lubricant is present in an amount of about 0.07% to about 1% by weight; the agglutinative is present in an amount of about 0.3% to 3% by weight; the stabilizer is present in an amount of about 1% to about 10% by weight; the plasticizer is present in an amount of about 0.1% to about 1% by weight.

A solution is prepared with the agglutinative. The excipient and about half of the lubricant are mixed, and then added to the saccharide and about half of the agglutinative solution. This mixture is formed into pellets that are dried in a drying stove. The water-soluble nutritional supplement is applied using the remainder of the agglutinative solution. After the application is completed, the pellets are dried in the drying stove.

A second, coating solution is then prepared using the stabilizer, plasticizer, and the other half of the lubricant; that is applied to the pellets to form the timed or retarded release pellets. These pellets are then dried. Assays are performed on the pellets and the timed or retarded release pellets in a solution mimicking the gastric pH and used to adjust the amounts of said formulations components to attain the desired timed or retarded release. In a specific embodiment, the nutritional supplement comprises glucosamine.

In another specific embodiment of the method of producing a composition of one or more pellets for a timed or retarded release capsule dosage of a water-soluble nutritional supplement form comprising at least one controlled release pellet. First, the water-soluble nutritional supplement and the formulation components are weighed such that the following proportions are present by weight: the nutritional supplement is present in about 60% to 95% by weight; the saccharide is present in about 1.5% to about 15% by weight; the excipient is present in about 0.6% to about 6% by weight; the lubricant is present in about 0.3% to about 3% by weight; the agglutinative is present in about 0.3% to about 3% by weight; and the plasticizer is present about 1.5% to about 12% by weight.

A solution is prepared with the agglutinative. The excipient and about half of the lubricant are mixed, and then added to the saccharide and about half of the agglutinative solution. This mixture is formed into pellets that are dried in a drying stove. The water-soluble nutritional supplement is applied using the remainder of the agglutinative solution. After the application is completed, the pellets are dried in the drying stove. A second coating solution is prepared using the plasticizer and the other half of the lubricant; that is applied to the pellets to form the timed or retarded release pellets. These pellets are then dried. Assays are performed on the pellets and the timed or retarded release pellets in a solution mimicking the gastric pH and used to adjust the amounts of said formulations components to attain the desired timed or retarded release. In a specific embodiment, the nutritional supplement comprises chondroitin.

The invention further provides a method of analyzing a composition of one or more pellets for a timed or retarded release capsule dosage of a glucosamine sulfate sodium chloride by performing chromatography. In a specific embodiment, multiple pellets prepared according to the invention and are placed into a hard gel capsule. The process is repeated until ten (10) such capsules have been prepared and are weighed individually. The average weight of their content is determined to be between about 1269.02 to about 1460 mg/capsule and the mean is determined. The relative standard deviation (RSD) is determined and is not more than about 6%. 20 mg of glucosamine sodium chloride is weighed and transferred quantitatively to a 25 mg volumetric flask. Water is then added to complete the volume and the solution is filtered through a 0.45 micron a high volume low pressure (HVLP) membrane and injected three times into a liquid chromatograph. The relative standard deviation is preferably not more than about 2%. 40 mg of glucosamine sulfate sodium chloride is weighed and transferred to a 50 ml volumetric flask. Water is added to complete volume and the solution is filtered through a 0.45 micron HVLP membrane and injected twice into a liquid chromatograph. The relative standard deviation is preferably not more than about 2%.

The content of a capsule is crushed and transferred to a 500 ml volumetric flask. 200 ml of water is added and the solution is placed in an ultrasonic Triturate for 15 minutes. Water is added to complete the volume and the solution is mixed well and filtered through a 0.45 micron HVLP membrane and injected once.

In view of the above description and the examples below, one of ordinary skill in the art will be able to practice the invention as claimed without undue experimentation. The foregoing will be better understood with reference to the following examples that detail certain procedures for the preparation of formulations according to the present invention. All references made to these examples are for the purposes of illustration. The following examples should not be considered exhaustive, but merely illustrative of only a few of the many embodiments contemplated by the present invention.

### EXAMPLE 1

The present example relates to a controlled release pelletized formulation of glucosamine sulfate, C₆H₁₃NO₅, beta-(1,4)-2-amino-2-deoxy-D-glucose, or poly-D-glucosamine, or poly N-acetyl-D-glucosamine. The formulation uses refined sugar as the saccharide, silicon dioxide as the excipient, talc as the lubricant, hydroxypropylmethylcellulose as the agglutinative, shellac gum as the stabilizing agent and diethyl phthalate in the following proportional weights:

| | |
|---|---|
| Glucosamine Sulfate | 88.00% |
| Refined Sugar | 4.97% |
| Silicon Dioxide | 1.80% |
| Talc | .22% |
| Hydroxypropylmethylcellulose | 1.00% |
| Shellac Gum | 3.66% |
| Diethyl phthalate | .35% |

A solution was prepared with the agglutinative. The excipient and about half of the lubricant were mixed, and then added to the saccharide and about half of the agglutinative solution. This mixture was formed into pellets that are dried in a drying stove. The water-soluble nutritional supplement was applied using the remainder of the agglutinative solution. After the application was completed, the pellets were , and were dried in the drying stove. A second, coating solution was then prepared using the stabilizer, plasticizer, and the other half of the lubricant; that was applied to the pellets to form the timed or retarded release pellets. These pellets were then dried. Assays were performed on the pellets and the timed or retarded release pellets in a solution mimicking the gastric pH and used to adjust the amounts of said formulations components to attain the desired timed or retarded release. Glucosamine released from the pellets was tested in a #2 paddle at 50 rpm in 900 ml of water at 37 degree C. +/−0.5 degrees.

TABLE 2

| Time (h) | Cumulative Percent Glucosamine |
|---|---|
| 1 | 19% |
| 4 | 59% |
| 8 | 81% |
| 12 | 88% |

FIG. 1 depicts the dissolution profile of the above Example:

[image]

### EXAMPLE 2

The present example relates to a controlled release pelletized formulation of chondroitin, its salts or esters, (C₁₄H₁₉NO₁₄SNa₂)ₙ; N-acetylchondrosamine (2-acetamide-2-deoxi-D-galactopiranose) and D-gluoronic acid copolymer. The formulation uses organic sucrose as the saccharide, silicon dioxide as the excipient, talc as the lubricant, hydroxypropylmethylcellulose as the agglutinative, and methacrylic acid copolymer as the retarding agent in the following proportional weights:

| | |
|---|---|
| Chondroitin Sulfate | 88.00% |
| Organic Sucrose | 4.97% |
| Silicon dioxide | 1.80% |
| Talc | 0.22% |
| Hydroxypropyl Methylcellulose | 1.00% |
| Methacrylic acid copolymer | 4.01% |

When the pellets dried, the active substance, chondroitin sulfate, using the hydroxypropyl methylcellulose solution as an agglutinative (ingredient that acts at this stage as a permeable agent or layer) was applied. Once the application of the active substance was completed, the pellets obtained were dried in the drying stove.

A solution was prepared with methacrylic acid copolymer as a retarding solution. The pellets were coated using the retarding solution and the other talc part, to obtain the time release pellets. The chondroitin sulfate time release pellets were then dried. The pellets were percolated, having a pellet measurement or particle size between 590 μm and 1190 μm. The pellets were then enclosed in capsules with different chondroitin sulfate concentrations required per capsules, such as: 100 mg, 200 mg, 250 mg, 300 mg, 400 mg, 500 mg and 600 mg.

Chondroitin release from the pellets was tested in a #2 paddle at 50 rpm in 900 ml of water at 37 degree C. +/−0.5 degrees.

TABLE 3

| Time (h) | Cumulative Percent Chondroitin |
|---|---|
| 1 | 30% |
| 4 | 56% |
| 8 | 88% |
| 12 | 96% |

FIG. 2, below, depicts the dissolution formula above:

[image]

### EXAMPLE 3

A core solution is prepared similarly to that described in Example 1, and glucosamine added. The coating solution is then prepared using the stabilizer, plasticizer, and the other half of the lubricant; that is applied to the pellets to form the timed or retarded release pellets and in half the quantity as in Example 1. Assays are performed on the pellets and the timed or retarded release pellets in a solution mimicking the gastric pH and used to adjust the amounts of said formulations components to attain the desired timed or retarded release. Glucosamine released from the pellets was tested in a #2 paddle at 50 rpm in 900 ml of water at 37 degree C. +/−0.5 degrees and it is found that 90% of the glucosamine is released after 12 hours, while the 1 and 4 hour release data was within the ranges seen in Example 1.

### EXAMPLE 4

The present example relates to the treatment of arthritis in humans by administering the composition described in Example 1. Twenty (20) patients with arthritis of the knee are administered the composition described in Example 1 at a dose of 500 mg twice a day, once upon awakening and once 12 hours later. Twenty-four (24) patients with osteoarthritis of the knee are administered matching placebo. The structural condition of the ankle is assessed by measuring the .alpha.-talocalcaneal angle by X-ray photography. The patients are asked to quantify their pain while performing various activities of daily living according to the Quebec Paid Disability Index. The activities are common ones such as getting up from bed, walking fifteen (15) minutes.

In clinical evaluation, a comparison is made in patients between before treatment and after one week of treatment using the Quebec index (total marks of the degree of lumbago answered by the patients from 0 mark to 5 marks in 20 items of daily life motion, the most serious symptoms being is 100 marks). The subjects are asked to rate their pain as: No pain at all 0 mark; Slightly difficult 1 marks; Sometimes difficult 2 marks; Frequently difficult 3 marks; Always difficult 4 marks; Cannot at all 5 marks. The Wilcoxon test was used for statistical testing.

There is a statisitically significant difference in the effect of the composition described in Example 1 and the matching placebo.

Green Coffee Extract

This embodiment relates to compositions comprising one or more pellets for a timed or retarded release of a water-soluble nutritional supplement mix in the stomach and/or gastrointestinal tract of a human, wherein said one or more pellets comprise: an admixture of (1) a water-soluble nutritional supplement mix in an amount of about 40% to about 90% by weight; (2) a saccharide in an amount of about 10% to about 30% by weight; (3) an excipient in an amount of about 1% to about 8% by weight; (4) one or more lubricants in a combined amount of about 1% to about 8% by weight; (5) an agglutinative and stabilizing agent in an amount of about 0.5% to about 10% by weight. In this embodiment, the agglutinative and stabilizing agent may be the same compound, specifically Shellac, though other polymers may be employed. A more specific embodiment is an admixture of (1) the water-soluble nutritional supplement mix in an amount of about 40% to about 88% by weight; (2) said saccharide in an amount of about 14% to about 46% by weight; (3) said excipient in an amount of about 1.4% to about 5.1% by weight; (4) said one or more lubricants in a combined amount of about 1.4% to about 5% by weight; (5) said agglutinative and stabilizing agent in an amount of about 0.9% to about 3.4% by weight. The pellets of the composition may be inside a hard capsule.

The composition of timed or retarded release water-soluble nutritional supplement mix is comprised of one or more selected from the group consisting of green coffee extract, vinpocetine, and L-carnitine. Optionally, the water soluble nutritional supplement mix is further comprised of one or more selected from the group consisting of amylase inhibitor, niacin, vitamin B3 (niacinamide), vitamin B6 (pyridoxine hydrocholoride), vitamin B12 (cyanocobaolamin), vitamin B5 (pantothenic acid), inositol, phosphorus, iodine, and chromium, and/or their dietary and nutraceutically acceptable salts of the same and/or hydrates of the active substance that provide a timed release formulation of the active substance.

In a more specific embodiment, the compounds and minerals that isolated from the green coffee extract include chlorogenic acid, boron, silicon, chromium, barium, tungsten, and bismuth, and most specifically may be isolated in the following amounts by weight:

(1) said caffeine in an amount of about 94%;

(2) said chlorogenic acid in an amount of about 1%;

(3) said B vitamins are present in an amount of about 2.5 mg/g total;

(4) said boron in an amount of about 5 ppm;

(5) said silicon in an amount of about 75 ppm;

(6) said chromium in an amount of about 3 ppm;

(7) said barium in an amount of about 1.3 ppm;

(8) said tungsten in an amount of about 5 ppm; and

(9) said bismuth in an amount of about 24 ppm.

In yet another embodiment of the invention, an immediate release powder is added to the controlled release pellets inside the capsule, to prepare an energy releasing composition. Specifically, the water-soluble supplement mix is green coffee extract and vinpocetine and the power is comprised of Yerba mate leaf extract (Ilex paraguariensis), ginseng, and taurine, and powdered green coffee extract, and most specifically are present in the following amounts:

(1) said green coffee extract in an amount of about 50 mg to about 300 mg;

(2) said vinpocetine in an amount of about 2 mg to about 15 mg;

(3) said niacin in an amount of about 10 mg to about 40 mg;

(4) said Vitamin B6 as Pyridoxine HCl in an amount of about 1 mg to about 4 mg;

(5) said Vitamin B12 in an amount of about 2 mcg to about 10 mcg;

(6) said Pantethoic acid in an amount of about 2 mg to about 10 mg;

(7) said Inositol in an amount of about 10 mg to about 60 mg;

(8) said Phosphorus in an amount of about 10 mg to about 40 mg;

said powder for immediate release is comprised of:

(1) said powdered green coffee extract for immediate release in an amount of about 50 mg to about 300 mg;

(2) said Yerba mate leaf extract (Ilex paraguariensis) in an amount of about 50 mg to about 300 mg;

(3) said ginseng in an amount of about 30 mg to about 200 mg;

(4) said taurine, in an amount of about 30 mg to about 150 mg.

Most specifically, the ingredients are present in the following amounts:

(1) said green coffee extract in an amount of about 100 mg;

(2) said vinpocetine in an amount of about 5 mg;

(3) said niacin in an amount of about 20 mg;

(4) said vitamin B6 as Pyridoxine HCl in an amount of about 2 mg;

(5) said vitamin B12 in an amount of about 5 mcg

(6) said pantethoic acid in an amount of about 5 mg;

(7) said inositol in an amount of about 30 mg;

(8) said phosphorus in an amount of about 20 mg;

said powder for immediate release is comprised of:

(1) said powdered green coffee extract for immediate release in an amount of about 150 mg;
(2) said Yerba mate leaf extract (Ilex paraguariensis) in an amount of about 125 mg;
(3) said ginseng in an amount of about 100 mg;
(4) said taurine in an amount of about 75 mg.

In another specific embodiment, said timed or retarded release water-soluble nutritional supplement mix is comprised of green coffee extract and L-carnitine selected from the group consisting of alkanoyl L-carnitine selected from the group consisting of acetyl, propionyl, butyryl, valeryl and isovaleryl L-carnitine, or a pharmaceutically acceptable salt thereof, and a pharmacologically acceptable excipient, vitamin B6 (pyridoxine hydrochloride), chromium picolinate, iodine, and inositol. Most specifically, the L-carnitine may be L-carnitine fumarate, tartrate, or chloride.

In yet another embodiment of the invention, an immediate release powder is added to the controlled release pellets inside the capsule, to prepare a weight control composition. Specifically, the water-soluble supplement mix is green coffee extract and L-carnitine fumarate and the power is comprised of green coffee extract, green tea extract (Camellia sinensis), cayenne extract, amylase inhibitors, and white willow extract (Salix alba). These ingredient are found in the following amounts:

(1) said green coffee extract present in an amount of about 50 mg to about 300 mg;

(2) said L-carnitine present in an amount of about 50 mg to about 300 mg;

(3) said vitamin B6 as pyridoxine HCl present in an amount of about 1 mg to about 4 mg

(4) said chromium picolinate present in an amount of about 10 mcg to about 200 mcg;

(5) said iodine present in an amount of about 10 mcg to about 100 mcg;

(6) said inositol present in an amount of about 10 mg to about 90 mg;

said powder for immediate release is comprised of:

(1) said green coffee extract in an amount of about 50 mg to about 300 mg
(2) said green tea leaf extract (Camellia sinensis) in an amount of about 50 mg to about 300 mg;
(3) said cayenne extract in an amount of about 20 mg to about 200 mg;
(4) said amylase inhibitor in an amount of about 30 mg to about 300 mg.
(5) said white willow extract (Salix alba)in an amount of about 5 mg to about 60 mg.

Most specifically, the ingredients are found in the following amounts:

(1) said green coffee extract in an amount of about 100 mg;

(2) said L-carnitine in an amount of about 100 mg.

(3) said vitamin B6 as Pyridoxine HCl in an amount of about 1.50 mg;

(4) said chromium in an amount of about 50 mcg;

(5) said iodine in an amount of about 38 mcg;

(6) said inositol is present in an amount of about 30 mg;

Said powder for immediate release is comprised of:

(1) said green coffee extract for immediate release in an amount of about 100 mg

(2) said green tea leaf extract (Camellia sinensis) in an amount of about 150 mg;

(3) said cayenne extract in an amount of about 60 mg;

(4) said amylase inhibitor in an amount of about 100 mg;

(5) said white willow extract in an amount of about 20 mg.

Optionally, the admixture may further be comprised of amylase inhibitor selected from the group consisting of wheat extract and phaseolamine from white bean extract, most specifically phaseolamine from white bean extract and in an amount of about 100 mg.

In a specific embodiment of the invention, the green coffee extract composition demonstrates the following the timed or retarded release dissolution profile when tested in a No. 2 (paddle) at 50 rpm in 900 ml of water at 37 degree C. +/−0.5 degree:

after 1 hour not more than 25% of said green coffee extract is released;

after 4 hours about 25% to about 60% of said green coffee extract is released; and

after 8 hours about 60% to about 85% of said green coffee extract is released;

after 12 hours not less than 85% of said green coffee extract is released.

Most specifically the green coffee extract composition demonstrates the following timed or retarded release dosage release pellet exhibits the following dissolution profile when tested in a No. 2 (paddle) at 50 rpm in 900 ml of water at 37 degree C. +/−0.5 degree:

after 1 hour about 12% of said green coffee extract is released;

after 4 hours about 50% of said green coffee extract is released;

after 8 hours about 81% of said green coffee extract is released; and

after 12 hours about 94% of said green coffee extract is released.

In a specific embodiment of the invention, the vinpocetine composition demonstrates the following timed or retarded release dissolution profile when tested in a No. 2 (paddle) at 50 rpm in 900 ml of water at 37 degree C. +/−0.5 degree:

after 1 hour about 20% to about 50% of said vinpocetine is released;

after 4 hours about 50% to about 80% % of said vinpocetine is released; and

after 8 hours about not less than 80% of said vinpocetine is released;

after 12 hours not less than 85% of said vinpocetine is released.

Most specifically, the vinpocetine composition demonstrates the following timed or retarded release dissolution profile when tested in a No. 2 (paddle) at 50 rpm in 900 ml of water at 37 degree C. +/−0.5 degree:

after 1 hour about 45% of said vinpocetine is released;

after 4 hours about 78% of said vinpocetine is released;

after 8 hours about 92% of said vinpocetine is released.

after 12 hours no less than 96.% of said vinpocetine is released.

In a specific embodiment of the invention, the L-carnitine composition demonstrates the following timed or retarded release dissolution profile when tested in a No. 2 (paddle) at 50 rpm in 900 ml of water at 37 degree C. +/−0.5 degree:

after 1 hour about 20% to about 45% of said L-carnitine is released;

after 4 hours about 40% to about 75% of said L-carnitine is released; and

after 8 hours about 60% to about 90% of said L-carnitine is released;

after 12 hours not less than 85% of said L-carnitine is released.

Most specifically, the L-carnitine composition demonstrates the following timed or retarded release dissolution profile when tested in a No. 2 (paddle) at 50 rpm in 900 ml of water at 37 degree C. +/−0.5 degree: after 1 hour about 38% of said L-carnitine is released;

after 4 hours about 64% of said L-carnitine is released; and

after 8 hours about 82% of said L-camitine is released;

after 12 hours not less than about 96% of said L-carnitine is released.

To prepare the powdered green coffee extract, an immediate release powder is later formulated into controlled release pellets by the methods disclosed above. To prepare the immediate release powder, the following steps are employed:

(a) green coffee beans are purchased;

(b) the green coffee beans are soaked to remove skin;

(c) the shelled green coffee beans are treated with with activated carbon and heat;

(d) the treated and shelled green coffee beans are filtered;

(e) the mixture is cooled and crystallized;

(f) the green coffee is inserted into a vacuum pipe;

(g) the green coffee is sent to a centrifuge with water and other natural solvents and rotated for several hours;

(h) a thick liquid is obtained in the centrifugation process;

(i) the thick liquid is separated from the coffee bean still inside the torpedo

(j) the thick liquid is sent to an open tank and combined with liquid, wax, powder, and mixture) and is left there for a period of time to sediment;

(k) the liquid is checked and analyzed when when wax and powder have sedimented,

(l) the water, now known as mother liquor, is resent to another torpedo to restart the decaffeination process with the coffee beans;

(m) the powder that remains in the tank is removed and naturally dried.

(n) the powder is sterilized

To prepare the timed or retarded release of formulation of the powdered green coffee extract for immediate release the following additional steps are employed:

(a) weighing the green coffee extract, additional water-soluble nutritional supplements, and formulation components, wherein said formulation components comprise a saccharide, an excipient, one or more lubricants, an agglutinative and a stabilizer, such that the following proportions are present by weight:

said water-soluble nutritional supplement mix in an amount of about 40% to about 90% by weight;

said saccharide in an amount of about 10% to about 30% by weight;

said excipient in an amount of about 1% to about 8% by weight;

said lubricants in an amount of about 1% to about 8% by weight; and

said agglutinative and stabilizer in an amount of about 0.5% to about 10% by weight;

(b) preparing a solution with said agglutinative;

(c) preparing a mixture with the excipient and half of the lubricant

(d) adding said mixture of excipient and lubricant to the saccharide and about one half of said solution of the agglutinative;

(e) forming said pellets from the mixture of step (d);

(f) drying said pellets;

(g) applying the green coffee extract with or without additional agglutinative solution;

(h) drying the pellets after the application is complete;

(i) preparing a solution using the stabilizer, agglutinative and the other half of the lubricant;

(j) applying solution of step (i) to the pellets to form the timed or retarded release pellets;

(k) drying the pellets;

(l) assaying the pellets and the timed or retarded release pellets in a solution of gastric pH; and

(m) adjusting the amounts of said formulations components to attain the desired timed or retarded release.

To prepare the formulations that contain two or more water-soluble nutritional supplements, specifically coffee with vinpocetine, and coffee with L-carnitine, which may also contain one or more vitamins selected from the list consisting of niacin, Vitamin B3 (niacinamide), vitamin B6 (pyridoxine hydrocholoride), vitamin B12 (cyanocobaolamin), vitamin B5 (pantothenic acid), inositol, and phosphorus, the steps a-m above are repeated as follows:
(a) a single supplement pellet is prepared and coated as described above;
(b) the second water-soluble nutritional supplement and the other components are weighed, and a solution is prepared with the second supplement and the agglutinative.
(c) the pellets are dried in the drying stove;
(d) a solution is prepared of the second water-soluble nutritional supplement with or without an agglutinative;
(e) the solution of the second water-soluble nutritional supplement with or without agglutinative is applied to the single layer pellets;
(f) a solution is then prepared using the stabilizer and the lubricant.
(g) the solution of the stabilizer and the lubricant is then applied to the pellets to form the timed or retarded release pellets.
(h) the two layered timed or retarded release pellets are dried;
(i) steps d-h are repeated for every layer of water-soluble nutritional supplement that is to be prepared.

In a specific embodiment of this composition, the first of said layers of water-soluble nutritional supplement mix is green coffee extract, vinpocetine, and L-carnitine and the second of said layers of nutritional supplement mix is comprised of one or more selected from the group consisting of vinpocetine and L-carnitine. In this embodiment, more specifically layer one is green coffee extract and Shellac gum and layer two is vinpocetine and methacrylic acid.

### EXAMPLE 5

The present example relates to a controlled release pelletized formulation of green coffee extract. The formulation uses organic sucrose as the saccharide, silicon dioxide as the excipient, talc as the lubricant, shellac gum as the agglutinative and stabilizing agent in the following proportional weights:

| | |
|---|---|
| green coffee extract | 40%-90% |
| organic sucrose | 10%-30% |
| silicon dioxide | 1%-8% |
| talc | 1%-8% |
| shellac gum | .5%-10% |

In a more specific embodiment, the green coffee extract is present in an amount of about 53%, the sucrose is present in an amount of about 36.3%, the silicon dioxide is present in an amount of about 3.86%, the talc is present in an amount of about 3.86% and the shellac gum is present in an amount of about 2.57%. This composition may be made using green coffee extract in an amount of about 100 mg or 300 mg, and the other ingredients proportionally added.

### EXAMPLE 6

The present example relates to a controlled release pelletized formulation of L-carnitine fumarate. The formulation uses organic sucrose as the saccharide, silicon dioxide as the excipient, talc as the lubricant, shellac gum as the agglutinative and stabilizing agent in the following proportional weights:

| | |
|---|---|
| L-carnitine fumarate | 72%-88% |
| organic sucrose | 14%-17% |
| silicon dioxide | 1.4%-1.8% |
| talc | 1.4%-1.8% |
| shellac gum | .99%-1.20% |

### EXAMPLE 7

The present example relates to a controlled release pelletized formulation of green coffee extract and L-carnitine fumarate. The formulation uses organic sucrose as the saccharide, silicon dioxide as the excipient, talc and magnesium stearate as the lubricants, shellac gum as the agglutinative and stabilizingP agent in the following proportional weights:

| | |
|---|---|
| green coffee extract | 15%-18% |
| L-carnitine fumarate | 38%-46% |
| organic sucrose | 29%-35% |
| silicon dioxide | 3.1%-3.7% |
| talc | 2.7%-3.3% |
| magnesium stearate | .3%-.4% |
| shellac gum | 2%-2.5% |

### EXAMPLE 8

The present example relates to a controlled release pelletized formulation of green coffee extract and vinpocetine. The formulation uses organic sucrose as the saccharide, silicon dioxide as the excipient, talc and magnesium stearate as the lubricants, shellac gum as the agglutinative and stabilizing agent in the following proportional weights:

| | |
|---|---|
| green coffee extract | 40%-48% |
| vinpocetine | .9%-1.1% |
| organic sucrose | 40%-46% |
| silicon dioxide | 4.2%-5.1% |
| talc | 2.9%-3.5% |
| magnesium stearate | 1.3%-1.6% |
| shellac gum | 2.8%-3.4% |

1. A composition comprising one or more pellets for a timed or retarded release of a water-soluble nutritional supplement mix in the stomach and/or gastrointestinal tract of a human, wherein said one or more pellets comprise: an admixture of
(1) a water-soluble nutritional supplement mix in an amount of about 40% to about 90% by weight;
(2) a saccharide in an amount of about 10% to about 60% by weight;
(3) an excipient in an amount of about 1% to about 8% by weight;
(4) one or more lubricants in a combined amount of about 1% to about 8% by weight;
(5) an agglutinative and stabilizing agent in an amount of about 0.5% to about 10% by weight.

2. The composition according to claim 1 wherein:
(1) said water-soluble nutritional supplement mix in an amount of about 40% to about 88% by weight;
(2) said saccharide in an amount of about 14% to about 46% by weight;
(3) said excipient in an amount of about 1.4% to about 5.1% by weight;
(4) said one or more lubricants in a combined amount of about 1.4% to about 5% by weight;
(5) said agglutinative and stabilizing agent in an amount of about 0.9% to about 3.4% by weight.

3. The composition according to claim 2 wherein said pellet(s) of said composition are inside a hard capsule.

4. The composition according to claim 1 wherein said water-soluble nutritional supplement mix is comprised of one or more selected from the group consisting of green coffee extract, vinpocetine, and L-carnitine.

5. The composition according to claim 4 wherein said water soluble nutritional supplement mix is further comprised of one or more selected from the group consisting of amylase inhibitor, niacin, vitamin B3 (niacinamide), vitamin B6 (pyridoxine hydrocholoride), vitamin B12 (cyanocobaolamin), vitamin B5 (pantothenic acid), inositol, phosphorus, iodine, and chromium, and/or their dietary and nutraceutically acceptable salts of the same and/or hydrates of the active substance that provide a timed release formulation of the active substance.

6. The composition according to claim 4, wherein said water-soluble nutritional supplement mix is green coffee extract and compounds and minerals that may be isolated from said green coffee extract include caffeine, chlorogenic acid, boron, silicon, chromium, barium, tungsten, and bismuth.

7. The composition according to claim 6 wherein said minerals may be isolated in the following amounts by weight:
(1) said caffeine in an amount of about 94%;
(2) said chlorogenic acid in an amount of about 1%;
(3) said B vitamins are present in an amount of about 2.5 mg/g total;
(4) said boron in an amount of about 5 ppm;
(5) said silicon in an amount of about 75 ppm;
(6) said chromium in an amount of about 3 ppm;
(7) said barium in an amount of about 1.3 ppm;
(8) said tungsten in an amount of about 5 ppm; and
(9) said bismuth in an amount of about 24 ppm.

8. The composition according to claim 4 wherein said water-soluble nutritional supplement mix is comprised of green coffee extract, vinpocetine, niacin, vitamin B3 (niacinamide), vitamin B6 (pyridoxine hydrocholoride), vitamin B12 (cyanocobaolamin), vitamin B5 (pantothenic acid), inositol, and phosphorus.

9. The composition according to claim 8 further comprising a powder for immediate release comprising Yerba mate leaf extract (Ilex paraguariensis), ginseng, and taurine, and powdered green coffee extract.

10. The composition according to claim 9 wherein said water-soluble nutritional supplement mix is comprised of:
(1) said green coffee extract in an amount of about 50 mg to about 300 mg;
(2) said vinpocetine in an amount of about 2 mg to about 15 mg;
(3) said niacin in an amount of about 10 mg to about 40 mg;
(4) said Vitamin B6 as Pyridoxine HCl in an amount of about 1 mg to about 4 mg;
(5) said Vitamin B12 in an amount of about 2 mcg to about 10 mcg;
(6) said Pantethoic acid in an amount of about 2 mg to about 10 mg;
(7) said Inositol in an amount of about 10 mg to about 60 mg;
(8) said Phosphorus in an amount of about 10 mg to about 40 mg;
said powder for immediate release is comprised of:
(1) said powdered green coffee extract for immediate release in an amount of about 50 mg to about 300 mg;
(2) said Yerba mate leaf extract (Ilex paraguariensis) in an amount of about 50 mg to about 300 mg;
(3) said ginseng in an amount of about 30 mg to about 200 mg;
(4) said taurine, in an amount of about 30 mg to about 150 mg.

11. The composition according to claim 10 wherein:
said water-soluble nutritional supplement mix is comprised of:
(1) said green coffee extract in an amount of about 100 mg;
(2) said vinpocetine in an amount of about 5 mg;
(3) said niacin in an amount of about 20 mg;
(4) said vitamin B6 as Pyridoxine HCl in an amount of about 2 mg;
(5) said vitamin B12 in an amount of about 5 mcg
(6) said pantethoic acid in an amount of about 5 mg;
(7) said inositol in an amount of about 30 mg;
(8) said phosphorus in an amount of about 20 mg;
said powder for immediate release is comprised of:
(1) said powdered green coffee extract for immediate release in an amount of about 150 mg;
(2) said Yerba mate leaf extract (Ilex paraguariensis) in an amount of about 125 mg;
(3) said ginseng in an amount of about 100 mg;
(4) said taurine in an amount of about 75 mg.

12. The composition according to claim 1 wherein said water-soluble nutritional supplement mix is comprised of green coffee extract, L-carnitine selected from the group consisting of alkanoyl L-carnitine selected from the group consisting of acetyl, propionyl, butyryl, valeryl and isovaleryl L-carnitine, or a pharmaceutically acceptable salt thereof, and a pharmacologically acceptable excipient, vitamin B6 (pyridoxine hydrochloride), chromium picolinate, iodine, and inositol.

13. The composition according to claim 12 wherein said pharmaceutically acceptable salt is selected from the group consisting of L-carnitine fumarate, tartrate, or chloride.

14. The composition according to claim 13 further comprising a powder for immediate release comprising green coffee extract, green tea extract (Camellia sinensis), cayenne extract, amylase inhibitors, and white willow extract (Salix alba).

15. The composition of claim 14 wherein said timed or retarded release water-soluble nutritional supplement mix is comprised of:
(1) said green coffee extract present in an amount of about 50 mg to about 300 mg;
(2) said L-carnitine a present in an amount of about 50 mg to about 300 mg;
(3) said vitamin B6 as pyridoxine HCl present in an amount of about 1 mg to about 4 mg
(4) said chromium picolinate present in an amount of about 10 mcg to about 200 mcg;
(5) said iodine present in an amount of about 10 mcg to about 100 mcg;
(6) said inositol present in an amount of about 10 mg to about 90 mg;
said powder for immediate release is comprised of:
(1) said green coffee extract in an amount of about 50 mg to about 300 mg
(2) said green tea leaf extract (Camellia sinensis) in an amount of about 50 mg to about 300 mg;
(3) said cayenne extract in an amount of about 20 mg to about 200 mg;
(4) said amylase inhibitor in an amount of about 30 mg to about 300 mg.
(5) said white willow extract (Salix alba) in an amount of about 5 mg to about 60 mg.

16. The composition according to claim 15 wherein said timed or retarded release water-soluble nutritional supplement mix is comprised of:
(1) said green coffee extract in an amount of about 100 mg;
(2) said L-camitine in an amount of about 100 mg.
(3) said vitamin B6 as Pyridoxine HCl in an amount of about 1.50 mg
(4) said chromium in an amount of about 50 mcg;
(5) said iodine in an amount of about 38 mcg;
(6) said inositol is present in an amount of about 30 mg;
Said powder for immediate release is comprised of:
(1) said green coffee extract for immediate release in an amount of about 100 mg;
(2) said green tea leaf extract (Camellia sinensis) in an amount of about 150 mg;
(3) said cayenne extract in an amount of about 60 mg;
(4) said amylase inhibitor in an amount of about 100 mg;
(5) said white willow extract in an amount of about 20 mg.

17. The composition according to claim 14 wherein said timed or retarded release water-soluble nutritional supplement mix is further comprised of amylase inhibitor selected from the group consisting of wheat extract and phaseolamine from white bean extract.

18. The composition according to claim 17 wherein said amylase inhibitor is phaseolamine from white bean extract and in an amount of about 100 mg.

19. The composition according to claim 6 wherein the timed or retarded release dosage release pellet exhibits the following dissolution profile when tested in a No. 2 (paddle) at 50 rpm in 900 ml of water at 37 degree C. +/−0.5 degree:
after 1 hour not more than 25% of said green coffee extract is released;
after 4 hours about 25% to about 60% of said green coffee extract is released; and
after 8 hours about 60% to about 85% of said green coffee extract is released;
after 12 hours not less than 85% of said green coffee extract is released.

20. The composition according to claim 19 wherein the timed or retarded release dosage release pellet exhibits the following dissolution profile when tested in a No. 2 (paddle) at 50 rpm in 900 ml of water at 37 degree C. +/−0.5 degree:
after 1 hour about 12% of said green coffee extract is released;
after 4 hours about 50% of said green coffee extract is released;
after 8 hours about 81% of said green coffee extract is released; and
after 12 hours about 94% of said green coffee extract is released.

21. The composition according to claim 8 wherein the timed or retarded release dosage release pellet exhibits the following dissolution profile when tested in a No. 2 (paddle) at 50 rpm in 900 ml of water at 37 degree C. +/−0.5 degree:
after 1 hour about 20% to about 50% of said vinpocetine is released;
after 4 hours about 50% to about 80% % of said vinpocetine is released; and
after 8 hours about not less than 80% of said vinpocetine is released;
after 12 hours not less than 85% of said vinpocetine is released.

22. The composition according to claim 21 wherein the timed or retarded release dosage release pellet exhibits the following dissolution profile when tested in a No. 2 (paddle) at 50 rpm in 900 ml of water at 37 degree C. +/−0.5 degree:
after 1 hour about 45% of said vinpocetine is released;
after 4 hours about 78% of said vinpocetine is released;
after 8 hours about 92% of said vinpocetine is released.
after 12 hours no less than 96.% of said vinpocetine is released.

23. The composition according to claim 12 wherein the timed or retarded release dosage release pellet exhibits the following dissolution profile when tested in a No. 2 (paddle) at 50 rpm in 900 ml of water at 37 degree C. +/−0.5 degree:
after 1 hour about 20% to about 45% of said L-carnitine is released;
after 4 hours about 40% to about 75% of said L-carnitine is released; and
after 8 hours about 60% to about 90% of said L-carnitine is released;
after 12 hours not less than 85% of said L-carnitine is released.

24. The composition according to claim 23 wherein the timed or retarded release dosage release pellet exhibits the following dissolution profile when tested in a No. 2 (paddle) at 50 rpm in 900 ml of water at 37 degree C. +/−0.5 degree:
after 1 hour about 38% of said L-camitine is released;
after 4 hours about 64% of said L-carnitine is released; and
after 8 hours about 82% of said L-carnitine is released;
after 12 hours not less than about 96% of said L-carnitine is released.

25. A method of producing a composition of powdered green coffee extract for immediate release comprising the steps of:
(a) green coffee beans are purchased;
(b) the green coffee beans are soaked to remove skin;
(c) the shelled green coffee beans are treated with activated carbon and heat;
(d) the treated and shelled green coffee beans are filtered;
(e) the mixture is cooled and crystallized;
(f) the green coffee is inserted into a vacuum pipe;
(g) the green coffee is sent to a centrifuge with water and other natural solvents and rotated for several hours;
(h) a thick liquid is obtained in the centrifugation process;
(i) the thick liquid is separated from the coffee bean still inside the torpedo
(j) the thick liquid is sent to an open tank and combined with liquid, wax, powder, and mixture) and is left there for a period of time to sediment;
(k) the liquid is checked and analyzed when wax and powder have sedimented,
(l) the water, now known as mother liquor, is resent to another torpedo to restart the decaffeination process with the coffee beans;
(m) the powder that remains in the tank is removed and naturally dried.
(n) the powder is sterilized.

26. A method of preparing one or more pellets for a timed or retarded release water-soluble nutritional supplement mix comprising producing a composition of powdered green coffee extract for immediate release according to claim 25; and further comprising the steps of:
(a) weighing the green coffee extract, additional water-soluble nutritional supplements, and formulation components, wherein said formulation components comprise a saccharide, an excipient, one or more lubricants, an agglutinative and a stabilizer, such that the following proportions are present by weight:
said timed or retarded release water-soluble nutritional supplement mix in an amount of about 40% to about 90% by weight;
said saccharide in an amount of about 10% to about 30% by weight;
said excipient in an amount of about 1% to about 8% by weight;
said lubricants in an amount of about 1% to about 8% by weight; and
said agglutinative and stabilizer in an amount of about 0.5% to about 10% by weight;
(b) preparing a solution with said agglutinative;
(c) preparing a mixture with the excipient and half of the lubricant
(d) adding said mixture of excipient and lubricant to the saccharide and about one half of said solution of the agglutinative;
(e) forming said pellets from the mixture of step (d);
(f) drying said pellets;
(g) applying the green coffee extract with or without additional agglutinative solution;
(h) drying the pellets after the application is complete;
(i) preparing a solution using the stabilizer, agglutinative and the other half of the lubricant;
(j) applying solution of step (i) to the pellets to form the timed or retarded release pellets;
(k) drying the pellets;
(l) assaying the pellets and the timed or retarded release pellets in a solution of gastric pH; and
(m) adjusting the amounts of said formulations components to attain the desired timed or retarded release.

27. A method of producing a composition of one or more pellets for a timed or retarded release capsule dosage of a water-nutritional supplement mix comprising powdered green coffee extract for immediate release according to claim 26 further comprising the additional steps of:
(a) a single supplement pellet is prepared and coated as described in claim 26;
(b) the second water-soluble nutritional supplement and the other components are weighed, and a solution is prepared with the second supplement and the agglutinative.
(c) the pellets are dried in the drying stove;
(d) a solution is prepared of the second water-soluble nutritional supplement with or without an agglutinative;
(e) the solution of the second water-soluble nutritional supplement with or without agglutinative is applied to the single layer pellets;
(f) a solution is then prepared using the stabilizer and the lubricant.
(g) the solution of the stabilizer and the lubricant is then applied to the pellets to form the timed or retarded release pellets.
(h) the two layered timed or retarded release pellets are dried;
(i) steps d-h are repeated for every layer of water-soluble nutritional supplement that is to be prepared.

28. A composition according to claim 27 wherein the first of said layers of water-soluble nutritional supplement mix is green coffee extract, vinpocetine, and L-carnitine and the second of said layers of nutritional supplement mix is comprised of one or more selected from the group consisting of vinpocetine and L-carnitine.

29. The composition according to claim 28 wherein said layers of water soluble nutritional supplement mix are further comprised of one or more selected from the group consisting of amylase inhibitor, niacin, Vitamin B3 (niacinamide), vitamin B6 (pyridoxine hydrocholoride), vitamin B12 (cyanocobaolamin), vitamin B5 (pantothenic acid), inositol, and phosphorus, inositol, phosphorus, iodine, and chromium.

30. A composition according to claim 29 wherein layer one is green coffee extract and Shellac gum and layer two is vinpocetine and methacrylic acid.
